(19) United States
(12) Patent Application Publication
(10) Pub. No.: US 2025/0360274 A1
(43) Pub. Date: Nov. 27, 2025

(54) INFUSION LEAK DETECTION SYSTEM AND METHODS

(71) Applicants: The Regents of the University of California, Oakland, CA (US); University of Notre Dame Du Lac, South Bend, IN (US); The Trustees of Indiana University, Bloomington, IN (US)

(72) Inventors: Thomas STAHOVICH, Riverside, CA (US); Nosang MYUNG, Notre Dame, IN (US); Kara Ellspermann GARCIA, Bloomington, IN (US)

(73) Assignees: The Regents of the University of California, Oakland, CA (US); University of Notre Dame Du Lac, South Bend, IN (US); The Trustees of Indiana University, Bloomington, IN (US)

(21) Appl. No.: 19/108,094

(22) PCT Filed: Sep. 1, 2023
(86) PCT No.: PCT/US2023/031909

§ 371 Date: Feb. 28, 2025

Related U.S. Application Data
(60) Provisional application No. 63/403,520, filed on Sep. 2, 2022.

(51) Int. Cl. A61M 5/50

(52) U.S. Cl. CPC A61M 5/5086; A61M 2202/07; A61M 2205/15; A61M 2205/18; A61M 2205/3327

(57) ABSTRACT

A system, method, and device for detecting leaks of a, drug at or near an infusion or injection site are disclosed. The system is configured to issue an alert upon detection of a specified chemical signature, such as a combination of gaseous analytes from a leaked therapeutic substance. The system can include a, sensor, such as one or more individual sensors or one or more sensor arrays; a multiplexer; a microcontroller unit; and a communications unit, such as a wireless communications unit.

### REFERENCE TO RELATED APPLICATIONS

This application claims priority to U.S. Provisional Application No. 63/403,520 that was filed on Sep. 2, 2022. The entire content of the application referenced above is hereby incorporated by reference herein.

### TECHNICAL FIELD

This document pertains generally, but not by way of limitation, to detection of gaseous chemical signatures.

### BACKGROUND

For patients with diabetes who use insulin pumps (>400,000 in the US alone and growing), a leak at an infusion site, tubing, or pump can go undetected until hyperglycemia (elevated blood glucose levels) is observed. Untreated hyperglycemia can result in a variety of acute complications including fatigue, nausea, blurred vision, headache, and inability to concentrate. Likewise, the failure of insulin delivery due to a leak can result in hypoinsulinemia, which in turn can lead to diabetic ketoacidosis, a potentially life-threatening complication.

Recent research (Hughes, MS et al., Frequency and Detection of Insulin Infusion Site Failure in the Type 1 Diabetes Exchange Online Community, Diabetes Technology & Therapeutics 2023 25:6. 426-430) suggests that 97% of pump users experienced infusion set failures—an estimated 5 million failures per year—and 41.4% of pump users experienced failures at least once per month. The latter group was significantly more likely to feel burned out by their diabetes technology and want to end pump use for different therapy, despite well-documented advantages in long-term diabetes management and outcomes (improvements in HbA1c levels and reduced incidence of diabetic ketoacidosis) related to pump usage. In an example, an infusion set can include at least one of the infusion site and associated tubing, such as tubing configured to transfer a fluid to the patient.

A common type of infusion set failure can include a leak at the infusion site. Currently, leak detection can require a patient or caregiver to smell, see, or feel wetness from leaking insulin around the infusion site. However, there are several impediments to detecting these kinds of leaks. First, the placement of an infusion site can make it difficult for the patient to detect a leak. For example, infusion sites can be placed on the lower back or buttocks and those sites are often covered with clothing. Second, individuals who regularly handle insulin can become “nose blind” to the smell. Third, leaks too small to produce detectable wetness can cause significant health problems Fourth, patients may be less likely to notice see, smell, or feel a leak during physical activity. Active individuals, including children and athletes, are particularly prone to this occurrence, as vigorous activity or swimming can cause adhesive loosening at the device-skin interface and dislodgment of the infusion cannula.

When physical signs are missed, a leak can be detected by elevated blood glucose levels (hyperglycemia). In these cases, patients can experience impaired cognition and performance resulting from hyperglycemia. Recovering from these symptoms can take several hours.

Current-generation insulin pumps can detect occlusion failures, such as in the case where the pump is unable to push out or deliver insulin. However, current pumps cannot detect leaks, such as in transferring insulin from the insulin pump to the patient at an infusion site. In one study of insulin infusion site failures, about 25% were detected by pump alarm (i.e., occlusion), 5.4% by smelling/seeing/feeling leaking insulin, 66.3% were detected by hyperglycemia or other adverse symptoms, and 3.2% were not detected at all.

An insulin leak, such as at the infusion site, in the tubing, or at the pump, can cause hyperglycemia which, left untreated, can lead to significant long-term complications such as nephropathy, retinopathy, diabetic neuropathy, diabetic ulcers, and the need for limb amputation. Hyperglycemia is associated with increased HbA1c levels and poor long-term health outcomes. Insulin leaks can also cause hypoinsulinemia, a serious complication that can deteriorate into diabetic ketoacidosis, a potentially life-threatening condition.

U.S. Pat. No. 9,696,291 (Gailius) mentions an electronic nose for determining the freshness of meat by analyzing volatile compounds and gases in the meat headspace and a method for determining meat freshness.

U.S. Pat. No. 10,422,771 (Kuroki) mentions an odor identification system including an operation array unit including at least two or more sensors, a sensor data processing unit, an odor factor information storing unit, and a pattern identification unit.

U.S. Pat. No. 10,592,510 (Amin) mentions systems and methods for a mobile electronic system that gathers and analyzes odors, airborne chemicals, and/or compounds.

### SUMMARY

The present inventors have recognized, among other things, that a problem to be solved can include detection of a leak, such as at an infusion site for a therapeutic drug. The present subject matter can provide a solution to this problem, such as by describing apparatus and methods to detect an insulin leak and alert a user to the presence of the leak.

In an example, the inventors have recognized a miniature noninvasive device, such as a leak alert device, can be used to alert patients and/or their caregivers to a leak associated with an infusion system. The device can include elements of an “electronic nose”, such as wearable sensor technology, to detect a leak, such as at an infusion site, based on the presence of a specific chemical or chemicals in proximity to the device, such as a chemical associated with an exogenous insulin. The device can alert the patient and/or caregiver to the presence of a leak, such as to indicate a need for corrective action to stop the leak before patient complications occur. A leak-indicating alert can lead to improved blood glucose management, which the landmark Diabetes Control and Complications Trial (DCCT Research Group: Diabetes Control and Complications Trial (DCCT): Update. Diabetes Care 1 Apr. 1990:13 (4): 427-433) demonstrated is critical for reducing the risk of chronic complications, such as eye, kidney, and nerve damage.

To address the issue of insulin leaks at the infusion site, a miniature, noninvasive device is needed to alert patients and/or their caregivers before the onset of elevated blood glucose. A detection system, such as to eliminate reliance on human smell and wetness detection, can provide peace of mind and improved quality of life for patients and caregivers living with an infusion device. It also has the potential to significantly improve short- and long-term health outcomes, such as for diabetic patients. In an example, improvement in the user experience related to insulin pumps has the potential to increase device adoption (as of 2018, 63% of individuals with type 1 diabetes use infusion pumps for the delivery of insulin) and further improve health outcomes in this population.

The innovation utilizes “electronic nose” (e-nose) or wearable gas sensor technology to detect an injection or infusion leak, such as an insulin leak based on insulin vapors. The sensor can detect one or more chemical constituents, such as insulin lispro, insulin lispro-aabc, insulin aspart, fast-acting insulin aspart (faster aspart), insulin glulisine, insulin human (regular insulin), neutral protamine Hagedorn (NPH) or isophane insulin, protamine zinc insulin, insulin glargine, insulin glargine-yfgn, insulin detemir, insulin human inhalation powder, arginine hydrochloride, dibasic sodium phosphate, disodium hydrogen phosphate dihydrate, disodium phosphate dihydrate, endogenous zinc, fumaryl diketopiperazine, glycerin, glycerol, hydrochloric acid, L-arginine, liraglutide, lixisenatide, magnesium chloride hexahydrate, mannitol, metacresol (m-cresol), methionine, niacinamide (vitamin B3), phenol, polysorbate 20, polysorbate 80, protamine sulfate, sodium chloride, sodium citrate dihydrate, sodium hydroxide, treprostinil sodium, tromethamine, zinc, zinc acetate, zinc oxide, zinc chloride, and water. For example, phenols are a class of semi-volatile organic chemicals. One or more of several specific phenols are contained in exogenous insulins and other pharmaceuticals. After a spill or leak, the smell of a phenol can be distinct and can be generally described as a medical or sterile smell. This smell can be strongest at the leak source, which could be at the infusion or injection site (common) or within the insulin pump and tubing (less frequent).

The present invention includes placement of a miniature e-nose sensor near the infusion or injection site, such as to detect when the concentration of vapor, such as a phenol vapor. exceeds a threshold, such as a user-selected threshold. A sensor can be used in several different embodiments including: (1) as a separate device attached to the side of an infusion pump, (2) as a feature incorporated into the pump device itself, (3) as a feature integrated into an infusion set, such as an insulin pump infusion set, and (4) as a standalone wearable device. Once a leak is detected, a user, such as the patient and/or caregiver, can receive an immediate notification, such as in the form of at least one of a blinking light, sound, or vibration produced by a wearable device. In an example, a notification can be sent to the user, such as at least one of the patient's or caregiver's smart phone or other computing device.

This summary is intended to provide an overview of subject matter of the present patent application. It is not intended to provide an exclusive or exhaustive explanation of the invention. The detailed description is included to provide further information about the present patent application.

### BRIEF DESCRIPTION OF THE DRAWINGS

FIG. 1 shows an example leak alert system.

FIG. 2 shows an example configuration of a sensor.

FIG. 3 shows an example system diagram of a microcontroller unit (MCU)

FIG. 4 shows an example of an operational state.

FIG. 5 shows an example method for using the leak alert system.

FIG. 6 shows a block diagram of an example machine.

FIG. 7 shows an example of an insulin leak alert system.

FIG. 8 shows an example of a sensor with multiple sensing elements.

In the drawings, which are not necessarily drawn to scale, like numerals may describe similar components in different views. Like numerals having different letter suffixes or subscripts may represent different instances of similar components. The drawings illustrate generally, by way of example, but not by way of limitation, various embodiments discussed in the present document.

### DETAILED DESCRIPTION

In the following description, for purposes of explanation, various details are set forth in order to provide a thorough understanding of some example embodiments. It will be apparent, however, to one skilled in the art that the present subject matter may be practiced without these specific details, or with slight alterations.

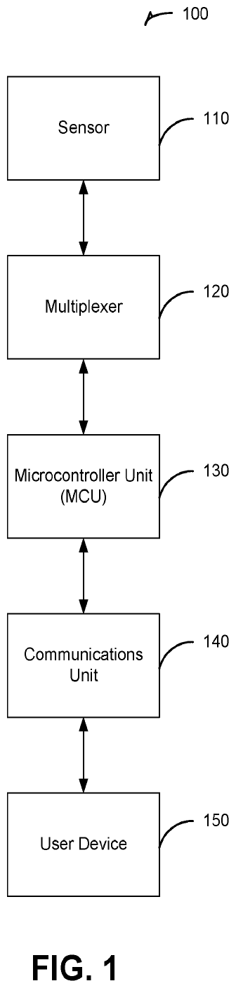

FIG. 1 shows an example of a leak alert system 100, such as a system configured to issue an alert upon detection of a specified chemical signature (i.e., a combination of one or more chemicals). The leak alert system 100 can include a sensor 110, such as one or more individual sensors or one or more sensor arrays; a multiplexer 120; a microcontroller unit (or MCU) 130; and a communications unit 140, such as a wireless communications unit.

The system 100 can detect insulin leaks by detecting gases emitted by the leaked insulin. By way of example, the system 100 can detect gasses produced by exogenous insulins such as: insulin lispro, including brand names Humalog® and Admelog®; insulin lispro-aabc, including brand name Lyumjev™; insulin aspart, including brand names Novolog® and Novorapid®; fast-acting insulin aspart (faster aspart), including brand name Fiasp®; insulin glulisine, including brand name Apidra®; insulin human (regular insulin), including brand names Humulin® R, Novolin® R, Velosulin® BR, Actrapid® Gensulin® R, and Myxredlin™; neutral protamine Hagedorn (NPH) or isophane insulin, including brand names Humulin® N, Novolin® N, Novolin® NPH, Gensulin® N, SciLin™ N, Insulatard ®, Protaphane®, and NPH Iletin® II; protamine zinc insulin, including brand name ProZinc®; insulin glargine, including brand names Basaglar®, Lantus®, Toujeo®, Rezvoglar™, and Soliqua®; insulin detemir, including brand name Levemir®; insulin degludec, including brand names Tresiba® and Xultophy®; insulin human inhalation powders, including brand names Afrezza® and Exhubera®; and combination, pre-mixed, or fixed combination insulins, including brand names Humalog Mix 75/25™, Humalog Mix 50/50™, Novomix® or Novolog® Mix 70/30. Novolin® 70/30, Humulin® 70/30, Humulin® 50/50, Gensulin® M30 (30/70), Ryzodeg® 70/30. The system 100 can detect these gasses by detecting one or more of their chemical constituents, such as insulin lispro, insulin lispro-aabc, insulin aspart, fast-acting insulin aspart (faster aspart), insulin glulisine, insulin human (regular insulin), neutral protamine Hagedorn (NPH) or isophane insulin, protamine zinc insulin, insulin glargine, insulin glargine-yfgn, insulin detemir, insulin human inhalation powder, arginine hydrochloride, dibasic sodium phosphate, disodium hydrogen phosphate dihydrate, disodium phosphate dihydrate, endogenous zinc, fumaryl diketopiperazine, glycerin, glycerol, hydrochloric acid, L-arginine, liraglutide, lixisenatide, magnesium chloride hexahydrate, mannitol, metacresol (m-cresol), methionine, niacinamide (vitamin B3), phenol, polysorbate 20, polysorbate 80, protamine sulfate, sodium chloride, sodium citrate dihydrate, sodium hydroxide, treprostinil sodium, tromethamine, zinc, zinc acetate, zinc oxide, zinc chloride, and water.

In an embodiment, the system 100 is an isolated device. For example, the sensor system 100 can take the form of a wristwatch, a keychain, a pendant, or other wearable form. In an embodiment, all or part of system 100 can be integrated into other devices, such as infusion pumps, infusion sets, and continuous glucose monitors. For example, the sensor 110 can be integrated into an insulin infusion set while the other elements of system 100 are integrated into an insulin pump or a separate device. The elements of system 100 can be communicatively linked via a direct connection, such as a wired connection, or via a wireless connection, such as a WiFi®, Bluetooth®, radiofrequency, or optical connection.

In an embodiment, one or more elements of system 100 can be reusable. In an embodiment, one or more elements of system 100 can be disposable. For example, the sensor 110 can be disposable while the other elements of system 100 can be reusable. In an embodiment, the sensor 110 can be integrated into the connector hub or adhesive patch of an infusion set so that the sensor is discarded with the infusion set when the latter is replaced by the patient. In an embodiment, the sensor 110 can comprise a sensor that is attached to or near the infusion site via an adhesive, an interlocking attachment, or similar means. Such an attachable sensor can be reusable or disposable.

The invention described herein includes a system and device for detecting leaks of a drug at an infusion or injection site. For example, the device can be used for detecting insulin leaks at an insulin infusion site for an insulin pump. However, it should be understood that the inventive system and device can be used for detecting any drug, medication, or therapeutic substance that is infused or injected into a patient (e.g., a human or other animal) via a needle, catheter, canula, or other infusion interface. In an embodiment, the inventive system and device can further be used for detecting leaks of drugs, medications, and therapeutic substances that are delivered intravenously, intramuscularly, subcutaneously, intrathecally, epidurally, or by other known routes of administration. While the invention is described herein as a system for detecting leaks at an infusion site, it should be understood that the system can detect leaks throughout the infusion apparatus, such as leaks at a hose, a pump, an O-ring, a seal, and other connections between insulin (or other drug) delivery system components. The system and device can also be used to detect a drug, medication, or therapeutic substance that is delivered by inhalation, such as inhaled insulins, powders, or other aerosols including bronchodilators used in treatment of asthma, as well as volatile substances including anesthetics and gases (e.g., oxygen, carbon dioxide, nitrous oxide), which may leak from the seal between delivery device (e.g., inhaler or oxygen mask) and the patient's nose and/or mouth.

In an embodiment, the system includes a wearable device for detecting leaks of a drug at an infusion site. In other embodiments, the system is a portable or stationary device that is placed in a hospital room, patient treatment room, or other locations in which drug infusions are administered. In an embodiment, the system can be integrated into other medical devices such as hospital beds, IV fluid pumps, and syringes. In an embodiment, the system includes a personal device configured to monitor leaks for a single patient. In another embodiment, the system includes an environmental monitor that is configured to detect leaks in one or more patients, such as one or more patients located in a particular space (e.g., a hospital room)

The system 100 can be located in proximity to a patient, such as a patient receiving a therapy requiring continuous titration of medicant with a medical device including an infusion pump or gravity infusion set. In an example, at least a portion of the system 100 can be located in proximity to the patient. For example, the sensor 110 can be attached to the patient, such as in proximity to a cannula inserted into the patient and associated with the infusion device, and other components of the system 100 can be located remotely from the patient, such as in wireless communication with the sensor 110.

The sensor 110 of system 100, can sense an indication of a chemical or chemicals and transform the sensed indication, such as into an electrical signal representing the indication of the sensed chemical. In an example, the electrical signal representing the indication can include a binary signal, such as to indicate the presence or absence of the chemical in proximity to the patient, or an analog signal, such as to represent a continuously variable indication over a range of values. For example, a continuously variable indication can include an indication of chemical concentration in proximity to the patient. The electrical signal can be transmitted to another component of the system 100, such as to at least one of the multiplexer 120, the MCU 130, or the communications unit 140.

In an example, the MCU 130 can include a processing module, such as to process the received electrical signal. The processor module can run an algorithm, such as an artificial intelligence (AI) or machine learning algorithm, to generate an algorithmic output, such as based at least in part on the received electrical signal. In an example, the MCU 130 can include an alarm module, such as to receive the algorithmic output. The alarm module can be configured to generate an alert, such as an alert signal, based at least in part on the received algorithmic output. An alert signal can be generated, such as when the received algorithmic output exceed a threshold value, such as target threshold value. The alert signal can be transmitted to another component of the system 100, such as to at least one of the MCU 130, a module of the MCU 130, or the communications unit 140. In an example, the alert signal can be received by the communications unit 140 and transmitted to a user device 150, such as via a wireless interface including, for example, Bluetooth®, ANT+ (“Advanced and Adaptive Network Technology,” a low-energy wireless protocol meant to collect and transfer sensor data), WiFi®, and RFID. In an example, the user device 150 can be used as a monitoring device to alert a user to an undesirable operational condition, such as a leak in proximity to the cannula associated with the infusion pump.

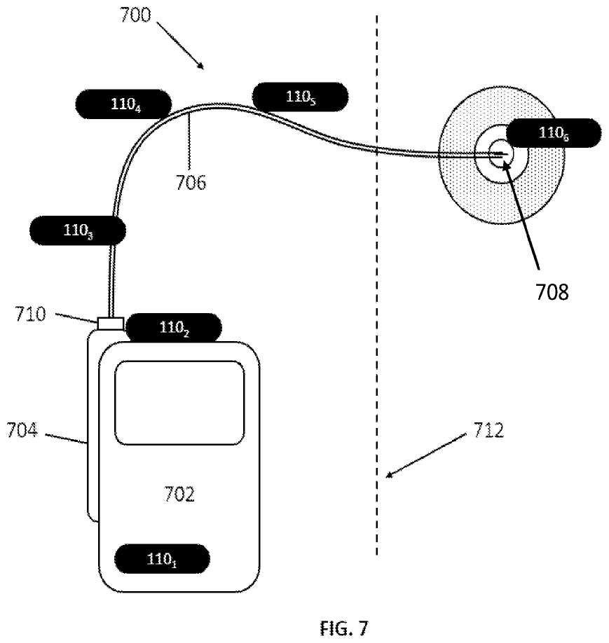

An example of an embodiment of leak alert system 100 is shown in FIG. 7, depicting a system 700 for detecting leaks from a continuous insulin infusion pump 702. A refillable insulin cartridge or reservoir 704 is connected to the insulin infusion pump 702. Insulin is delivered from the insulin cartridge 704 through tubing 706 into an infusion site 708, where a cannula (e.g., small steel or Teflon) extends beneath the skin to deliver insulin subcutaneously Leaks may occur at any point along system 700. For example, leaks can occur at the interface of the insulin cartridge 704 and the insulin infusion pump 702, at the interface of the insulin infusion pump 702/cartridge 704 and the tubing 706 (e.g., at O-ring 710), anywhere along the tubing 706 (e.g., if a hole exists or is introduced), at the interface of the tubing 706 and the infusion site 708, or between the cannula and the skin (i.e., infusion site). Although FIG. 7 includes tubing 706, it should be noted that system 700 can also work with tubeless insulin infusion systems.

Sensors 110₁₋₆ can be placed at various locations within system 700 to detect a leak in proximity to the sensor. Examples of sensor placements are shown FIG. 7. Sensor detection may depend, at least in part, on the sensitivity of the sensor, the distance by which the chemicals emitted from the leak are able to diffuse and/or convect, and the presence of semipermeable or impermeable barriers 712, such as clothing or secondary tape. In the case of high sensitivity and broad diffusion/convection, a single sensor 110 placed within or near the system may be sufficient to detect a leak at any point. Alternately, multiple sensors 110₁₋₆ can be placed in close vicinity to various locations where a leak may occur, as shown in FIG. 7. Additionally, inclusion of multiple sensors 110₁₋₆ can be used to identify the approximate location of the leak (e.g., via triangulation). For example, a leak with strongest signal at sensor 110₂ can indicate a leak at the O-ring 710, whereas a leak with strongest signal at sensor 110₆ can indicate a leak at the infusion site. A leak detected with strongest signal at sensors 110₄ or 110₅ can indicate a leak in the tubing 706 between sensors 110₄ and/or 110₅. In other scenarios, such as triangulating the source of the leak in a large hospital room, the same concept may be applied by spacing multiple sensors over much longer distances.

### Sensor

Referring again to FIG. 1, the sensor 110 can detect a gaseous chemical, such as a chemical vapor, in an environment surrounding a patient, such as a gaseous environment in contact with the skin of the patient, an infusion site, an infusion set, or an infusion pump The sensor 110 can also detect gaseous chemicals in an environment that is remote from the patient, infusion site, infusion set, or infusion pump. In an example, the gaseous chemical can come from a chemical unmetabolized by the patient, such as due to a drug that has leaked at the site of a cannula inserted into the patient. In an example, the gaseous chemical can come from a exogenous insulin that has leaked from an infusion site, an infusion set, or an insulin pump. The detection of gaseous chemicals produced by exogenous insulin, can indicate a leak of exogenous insulin from an infusion site, infusion system, or infusion pump. In an example, the gaseous chemical or chemicals can be from a leak of drugs, medications, and therapeutic substances that are delivered intravenously, intramuscularly, subcutaneously, intrathecally, epidurally, or by other known routes of administration. The detection of gaseous chemicals produced by drugs, medications, and therapeutic substances, can indicate a leak of such substances from an infusion site, infusion system, or infusion pump.

In an example, the sensor 110 can detect an indication of a gaseous chemical including a gaseous chemical from at least one of the following: insulin lispro, insulin lispro-aabc, insulin aspart, fast-acting insulin aspart (faster aspart), insulin glulisine, insulin human (regular insulin), neutral protamine Hagedorn (NPH) or isophane insulin, protamine zinc insulin, insulin glargine. insulin glargine-yfgn, insulin detemir, insulin human inhalation powder, arginine hydrochloride, dibasic sodium phosphate, disodium hydrogen phosphate dihydrate, disodium phosphate dihydrate, endogenous zinc, fumaryl diketopiperazine, glycerin, glycerol, hydrochloric acid. L-arginine, liraglutide, lixisenatide, magnesium chloride hexahy drate, mannitol, metacresol (m-cresol), methionine, niacinamide (vitamin B3), phenol, polysorbate 20, polysorbate 80, protamine sulfate, sodium chloride, sodium citrate dihydrate, sodium hydroxide, treprostinil sodium, tromethamine, zinc, zinc acetate, zinc oxide, zinc chloride, and water.

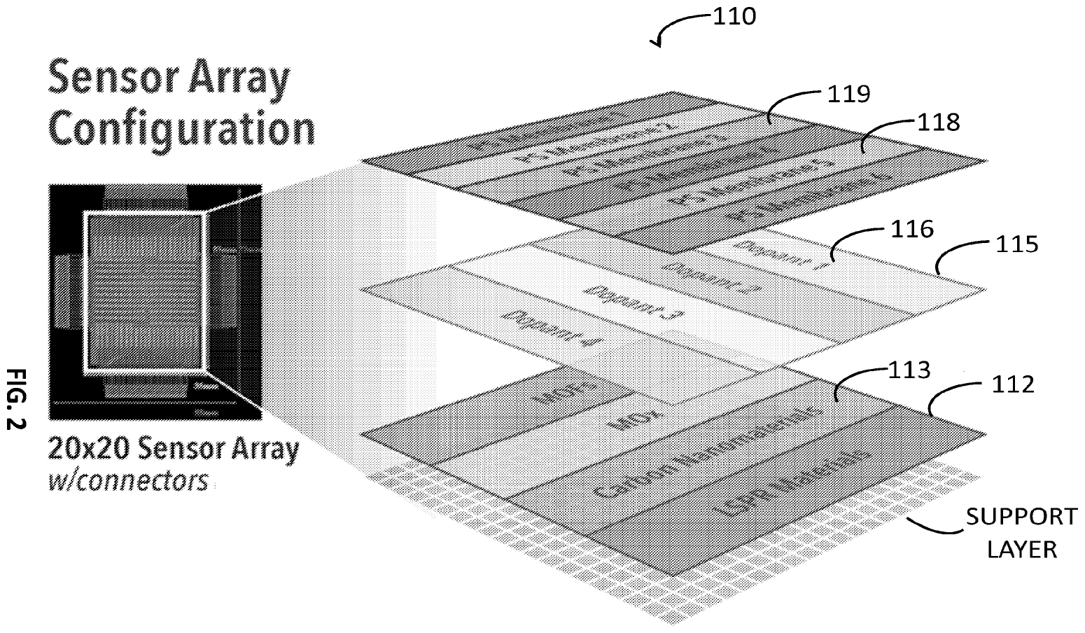

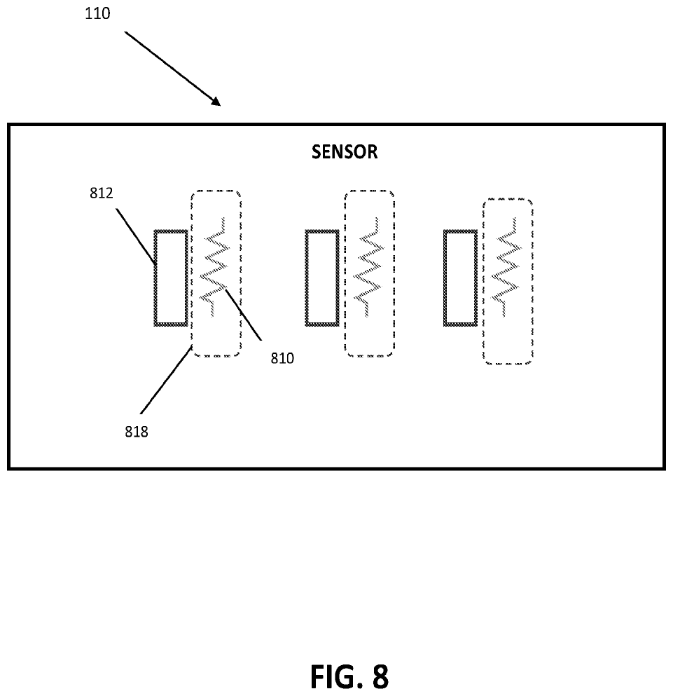

In an embodiment, the sensor 110 comprises one or more sensing elements 810, as shown in FIG. 8. In another embodiment, the sensor 110 comprises an array of sensing elements (shown in FIG. 2) with varying sensing characteristics. The sensor 110 comprises a sensing element 810. The sensor 110 can further comprise a heating element 812 located in proximity to the sensing element, such that the heating element 812 can control the temperature of the sensing element 810, thus controlling the sensing characteristics, such as sensitivity to specific chemicals, of the sensing element 810. In an embodiment, the sensing element 810 is enclosed in, surrounded by, or in located in proximity to a perm-selective membrane 818 configured to restrict the adsorption of gaseous molecules into the sensing element, thus enhancing the sensitivity and/or selectivity of the sensing element to a specific molecule or molecules. In a sensor array, different sensing elements can have different perm-selective membranes.

The sensing elements 810 in sensor 110 can produce signals, such as an electrical signals representative of the one or more gaseous chemicals or a chemical signature. In an example. adsorption of a gaseous chemical onto a surface of the sensing element 810 can change a property of the sensing element 810, such as an electrical property of the sensing element 810. An electrical property can include at least one of an electrical impedance, an electrical capacitance, or an electrical resistance. The change in an electrical property can indicate a characteristic of a gaseous chemical in the environment, such as the gaseous environment surrounding the patient. In an example, a characteristic of a gaseous chemical in the environment can include at least one of the presence or absence of the gaseous chemical in the environment, a concentration of the gaseous chemical in the environment, a change in concentration of the gaseous chemical in the environment, or a rate of change of concentration of the gaseous chemical in the environment.

The sensor 110 can comprise a chemiresistive sensor, such as a sensing element 810 configured to change its electrical resistance in response to the presence of an analyte (e.g., a gaseous chemical), such as a target analyte in a gaseous environment. For example, the sensing element 810 can be configured to change its electrical resistance in response to the binding of the target analyte to the sensing element 810. In an example, the sensing materials in sensing element 810 can include at least one of a metal oxide semiconductor (MOS), a carbon material, a metal organic framework (MOF), a covalent organic framework (COF), a phyllosilicate, a conducting polymers (CP), a transitional metal dichalcogenide (TMDC), and Mxene.

In an embodiment, the perm-selective barrier can include at least one of a polymer barrier including a molecular imprinted polymer: an inorganic barrier, including metal oxides; a molecular organic framework (MOF) barrier; and/or a covalent organic framework (COF) barrier. The perm-selective barrier can be selected to enhance at least one of selectivity or sensitivity of the sensor element for a specific analyte molecule or molecules.

The sensor 110 can comprise a single a single sensor 110 or a plurality of sensors 110. A sensor 110 can contain a single sensing element or an array of sensing elements. In an embodiment, a single sensing element is sensitive to one or more target analytes, such as exogenous insulins or components of exogenous insulins. In another embodiment, an array of sensing elements includes multiple sensing elements sensitive to target analytes, such as exogenous insulins or components of exogenous insulins. In another embodiment, an array of sensing elements includes one or more sensing elements sensitive target analytes, such as exogenous insulins or components of exogenous insulins, and one or more sensing elements sensitive to other environmental chemicals, such as to enable the target analytes to be more accurately distinguished from other environmental chemicals.

FIG. 2 shows an exploded view of an example configuration of a sensor 110, such as sensor containing a sensor array with a perm-selective barrier. The sensor 110 can include a primary sensing material layer 112 with a first surface 111 (not shown) in contact with a support layer and a second surface 113 opposite the first surface 111. The sensor can include a secondary sensing material layer 115 with a third surface 114 (not shown) facing the second surface 113 and a fourth surface 116 opposite the third surface 114. In an example, the second surface 113 can be in continuous contact with the third surface 114. The sensor 110 can include a perm-selective membrane 118 with a fifth surface 117 (not shown) facing the fourth surface 116 and a sixth surface 119 opposite the fifth surface 117. In an example, the fourth surface 116 can be in continuous contact with the fifth surface 117. In an example, the sensor 110 can include a device to sense a change in impedance, resistance, and capacitance, such as an impedimetric sensor.

The sensing material layer, such as at least one of primary sensing material layer 112 or secondary sensing material layer 115, can include a sensing material. In an example, the sensing material can include at least one of a metal oxide semiconductor (MOS), a carbon material, a metal organic framework (MOF), a covalent organic framework (COF), a phyllosilicate, a conductive polymer (CP), a transitional metal dichalcogenide (TMDC), and Mxene. The sensing material layer can be doped, such as with a dopant molecule to change a property of the sensing material layer. In an example, a dopant molecule can include another sensing material, such as a macromolecule and noble metals. In an example, the dopant molecule can be a molecule other than a metal oxide semiconductor (MOS), a carbon material, a metal organic framework (MOF), a covalent organic framework (COF), a phyllosilicate, a conductive polymer (CP), a transitional metal dichalcogenide (TMDC), and Mxene.

The sensor 110 can include a sensing element or sensor array configured to sense an environment in proximity to the sensor 100, such as, for example, within a radius of about one to 10 meters (about 1 m to 10 m) from the sensor. In an example, the sensor 110 can be located in proximity to an infusion set, such as on top of an infusion set attached to a patient. The sensor 110 can be integrated into a sensor system, such as a sensor system configured to detect chemical vapors from drugs, medications, and therapeutic substances leaked as they are administered to a patient. In an example, the sensor system 100 and or the sensor 110 can take the form of a wristwatch, a keychain, a pendant, or other wearable form.

### Multiplexer

In an embodiment, a multiplexer 120 is used to transmit electrical signals form the sensor 110 to the MCU 130. In an embodiment, the electrical signals from the sensor 110 can be directly transmitted to the MCU 130 without use of a multiplexer 120. The multiplexer 120 can include a switching device, such as implemented as at least one of a hardware device including an electronic circuit or a software module running on a computing device, such as including an MCU 130. The multiplexer 120 can include multiple input channels, such that there is a separate input channel for each electrical signal from sensor 110. The multiplexer 120 can include one or more output channels which are connected to input channels on the MCU 130. The multiplexer 120 is controlled by a logic signal, such as a logic signal from MCU 130, that causes one or more input channels of the multiplexer 120 to be connected to one or more output channels of the multiplexer. In an embodiment, the multiplexer 120 produces one-to-one connections, in which at most one input channel of MCU 130 is connected to any given output channel of multiplexer 120 at any given time. In an embodiment, the multiplexer 120 has one input channel for each electrical signal from sensor 110, and a single output channel connected to MCU 130. A logic signal from MCU 130 can enable MCU 130 to sequentially select and receive the electrical signals from sensor 110, one at a time, for further processing.

### Microcontroller Unit (MCU)

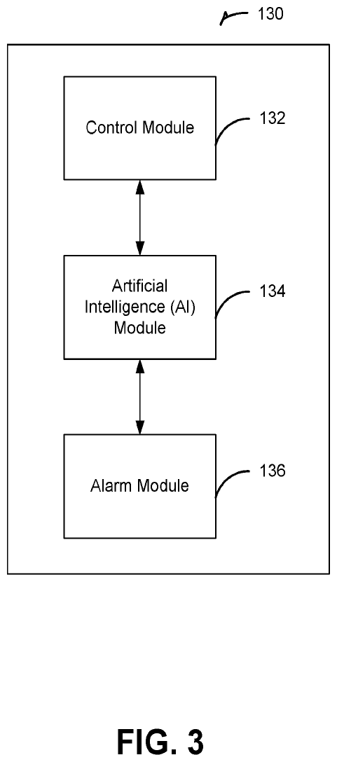

FIG. 3 shows an example system diagram of an MCU 130. The MCU 130 can include at least one of circuitry or software running on the circuitry, such as to facilitate operation of the system 100. In an example, the MCU 130 can include at least one of a control module 132, an artificial intelligence (AI) module 134, or an alarm module 136.

The control module 132 controls operation of the system 100. In an example, operational control of the system 100 can include at least one of controlling the sensor 110, such as activating and deactivating the sensor 110, and supplying control signals, such as a signal controlling the temperature of a heater or heaters integrated into the sensing elements of sensor 110: measuring electrical properties such as measuring the electrical impedance, electrical capacitance, or electrical resistance of sensing elements in the sensor 110; managing data, such as reading, digitizing, processing, and transmitting electrical signals received from the sensor 110, managing communication, such as communication between one or more components of the system 100; and managing power of the system 100. In an example, the control module 132 can provide a signal, such as to control a sensing element. In an example, the control module 132 can receive and measure a signal, such as an output from the sensor 110. In an example, the control module 132 can control system power, such as system power required to operate the system 100. In an example, the control module 132 digitizes the electrical signals from sensor 110 and provides the digitized signal data to the artificial intelligence module 134.

The artificial intelligence module 134 can process the electrical signals from the sensor 110 to identify target analytes. In the first step of processing, a feature extraction module (not shown) of the artificial intelligence module 134 can compute features from electrical signals received from the sensor 110. As an example, these features can include one or more of a peak signal value, the time to reach the peak signal value, the rate of change of the signal value, the difference between the signal value and a baseline value, a time-sequence of digitized values. parameters of a curve fit to the time-sequence of the signal values, and the frequency content of the time-sequence of the signal values.

Features from the feature extraction module can be transmitted to a machine learning module (not shown) of the artificial intelligence module 134. The machine learning module can compute a label from the features. In an example, the label includes one or more of a value characterizing the existence or absence of a target chemical, a value characterizing the concentration of a target chemical, a value characterizing the change of the concentration of a target chemical, a value characterizing the rate of change of the concentration of a target chemical, a value characterizing the difference between the concentration of a target chemical and the expected concentration of that chemical in the absence of a drug leak, and a value characterizing the presence or absence of a leak of a target drug. The label can comprise binary values, such as binary values indicating the presence or absence of target analytes, continuous values such as continuous values indicating the concentration of target analytes, or a combination of binary and continuous values.

In an embodiment, the machine learning module can employ one or more trained machine learning models. A machine learning model can comprise, for example, an artificial neural network, a recurrent neural network, a convolutional neural network, a decision tree, a random forest model, a regression model, a deep learning model, a k-nearest neighbor model, or other classification or regression model.

In an embodiment, the machine learning models in the machine learning module can be trained using training data obtained in a laboratory environment. The training data can comprise both positive and negative training examples. The positive training examples are examples of the response of sensor 110 when exposed to the target chemical, such as insulin. The negative examples are examples of the response of sensor 110 when exposed to non-target chemicals that may be in the environment such as household cleaners, perfumes, soaps, etc. The positive training examples can also include examples of the response of sensor 100 when exposed to both the target chemical, such as insulin, and non-target chemicals that may be in the environment such as household cleaners, perfumes, soaps, etc. Each of the training examples is assigned a label corresponding to true characteristics of the examples. For instance, a positive example is labeled as such and a negative example is labeled as such. Herein, there assigned labels are referred to as the true labels.

During the training of a machine learning model, the training examples can be input into the machine learning model and the labels output by the model can be recorded. An error can be computed for each training example. This error can comprise the difference between the true label of the training example and the label computed by the machine learning model. These errors can then be used to adjust the parameters of the machine learning model, such as, for example, the weights of an artificial neural network, to minimize the error. In an embodiment, the training processes is applied repeatedly to minimize the error. In an embodiment, the training data can be divided into a training set and a testing set such that the training set is used to adjust the parameters of the machine learning model and the testing set is used to evaluate the performance of the machine learning model where performance is characterized by one or more of accuracy, precision, recall, f-measure, mean absolute error, mean squared error, root mean squared error, or other similar performance measure. In an embodiment, the training process is conducted on a separate computer and the trained models are transferred to and stored in the artificial intelligence module 134 in the form of machine-readable data and computer executable instructions. In an embodiment, additional training to improve the performance of the machine learning model may occur during ordinary operation of the system 100 as the user provides feedback to system about false negative or false positive alarms.

Training the machine learning models using both positive and negative training examples enables the system 100 to distinguish between a target analyte and other analytes that may be found in the environment. Additionally, as described above, the sensor 110 can comprise a sensor array containing both sensing elements that are sensitive to the target analytes and sensing elements that are sensitive to other environmental analytes. Such a sensor design can enhance the ability of the machine learning models to distinguish target analytes from non-target analytes.

In an embodiment, the machine learning technique can include at least one of a classifier, such as a classifier to distinguish the presence of insulin in the environment, and a regression model, such as a regression model to quantify the concentration of insulin in the environment.

In an embodiment, the alarm module 136 receives as input the labels computed by the artificial intelligence module 134 and produces as output an alarm signal indicating the presence or absence of an insulin leak. The labels from the artificial intelligence module 134 indicate the presence or absence of target analytes and/or the concentration of target analytes. In an example, the alarm signal has a value of “1” to indicate the presence of a leak and a value of “0” to indicate the absence of a leak.

In an embodiment, the alarm module 136 identifies a leak as a state in which one or more target analytes is present. In an embodiment, the alarm module 136 identifies a leak as a state in which one or more target analytes is present for a duration of time that exceeds a threshold. In an embodiment, the alarm module 136 identifies a leak as a state in which the concentration of or one or more target analytes exceeds one or more thresholds. In an embodiment, the alarm module 136 identifies a leak as a state in which the concentration of or one or more target analytes exceeds one or more thresholds for a duration of time that exceeds a threshold. In an embodiment, the alarm module 136 identifies a leak as a state in which the concentration of or one or more target analytes increases at a rate exceeding a threshold. In an embodiment, the alarm module 136 identifies a leak as a state in which the concentration of or one or more target analytes increases at a rate exceeding a threshold for a duration of time that exceeds a threshold.

In an embodiment, the alarm module 136 indicates the absence of a leak during an interval of time immediately after the pump and/or infusion set is prepared for use by the patient such that insulin spilled and or released during the preparation of the insulin pump and/or infusion set does not cause false positive indications of leaks. For example, in some cases an insulin cartridge is filled with insulin by means of a syringe. In some cases, air bubbles are removed from such a syringe by inverting the syringe and expelling the bubbles by depressing the plunger on the syringe. In an embodiment, the alarm module 136 indicates the absence of a leak during an interval of time beginning with the start of the cartridge preparation and continuing for a preset duration. Similarly, before an infusion set is applied to a patient, it is frequently necessary to prime the infusion set by pumping insulin through it until the air is removed. In an embodiment, the alarm module 136 indicates the absence of a leak during an interval of time beginning with the start of the infusion set priming and continuing for a preset duration. In an embodiment, the alarm module 136 communicates with the insulin pump to determine the times at which the insulin pump is prepared for use, insulin cartridges are filled, and infusion sets are primed.

In an embodiment, the alarm module 136 contains an expected profile for the concentration or existence of one or more target analytes during the normal operation of an insulin pump and infusion set during a cycle of treatment. A cycle of treatment begins with the preparation, filling, and priming of the insulin pump, insulin cartridge, and infusion set and application of the infusion set to the patient. A cycle of treatment ends when then infusion is replaced or when the insulin pump and/or insulin cartridge is refilled or replaced. In an embodiment, the alarm module 136 indicates a leak when the concentration or presence of target analytes exceeds the expected profile for a duration of time exceeding a threshold.

The alarm module 136 can be configured to indicate a transition of a signal received from at least one of the sensor 110, multiplexer unit 120, control module 132, or artificial intelligence module 134, such as transition from a first operational state to a second operational state. An operational state can be defined by a threshold value, such as at least one of a minimum threshold value and a maximum threshold value. In an example, transition from a first operational state to a second operational state can be indicated by a change in an alarm signal value, such as a change in a memory register state on the MCU 130. In an example, the alarm signal value can assume a value of “0,” such as during operation of the system 100 in the first operational state. In an example, the alarm signal value can assume a value of “1,” such as during operation of the system 100 in the second operational state.

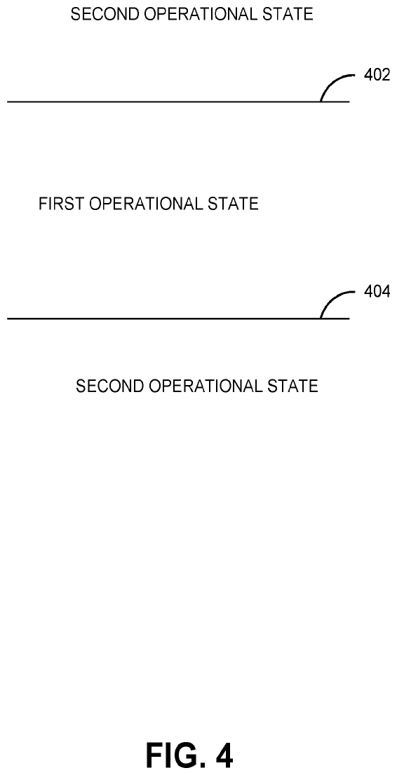

FIG. 4 shows an example of an operational state, such as a first operational state defined by an area between a minimum threshold value 402 and a maximum threshold value 404. In an example, a second operational state can include the area not included in the first operational state.

During operation of the system 100, a signal received from at least one of the sensor 110, multiplexer unit 120, control module 132, or artificial intelligence module 134 can be initially located in the first operational state, such as a value between the minimum threshold value 402 and the maximum threshold value 404. As the received signal varies with time, such as due to the sensor 110 detecting a change in a chemical signature or combination of gaseous chemicals, the received signal level can cross a threshold, such as to function in the second operational state. In an example, the received signal can increase, such as to have a value greater than the maximum threshold value 404 and the system 100 can transition from the first operational state to the second operational state. Likewise, while the system 100 is in the second operational state, the received signal can decrease, such as to have a value less than the maximum threshold value 404, and the system 100 can transition from the second operational state to the first operational state.

Referring again to FIG. 3, the alarm module 136 can be configured to assess transition of a received signal, such as to indicate at least one of a type or criticality of the received signal transition. In an example, the alarm module 136 can indicate the type of received signal transition, such as at least one of a sustained received signal transition that can indicate the presence of a continuous exogenous insulin leak, or a time-varying received signal transition that can indicate the resolution of an exogenous insulin lead, or a false alarm based on the presence of insulin not associated with a monitored infusion set.

The alarm module 136 can be configured with a threshold, such as an adjustable threshold. In an example, the adjustable threshold can be set by a user, such as by a patient, to a level selected to minimize the occurrence of a “false alarm,” such as due to trace amounts of exogenous insulin on the clothes of the user In an example, the adjustable threshold can be set by the MCU 130, such as by running software on the MCU 130 that can sense and track background insulin level in an environment and adjust threshold level based on historical background insulin level in the environment.

In addition to detecting and alerting the difference between two states, the alarm module 136 can provide additional detail such as whether the signal is sustained (e.g., continuous leak) or time-varying (indicating problem resolution, or false alarm based on presence of nearby insulin), as well as the onset and duration of the signal

### Communications Unit

The communications unit 140 can allow signals to be transmitted, such as to components of the system 100 or to devices separate from the system 100. In an example, the communications unit 140 can be used to transmit signals to components of the system 100, such as from the sensor 110 to at least one of the multiplexer 120 or the MCU 130. In an example, the communications unit 140 can transmit a signal to a device separate from the system 100, such as an alert signal from the MCU 130 to an on-the-go (OTG) device, including a cell phone. In an example, the communications unit 140 can transmit a signal to a device, such as a medical device including an insulin infusion pump.

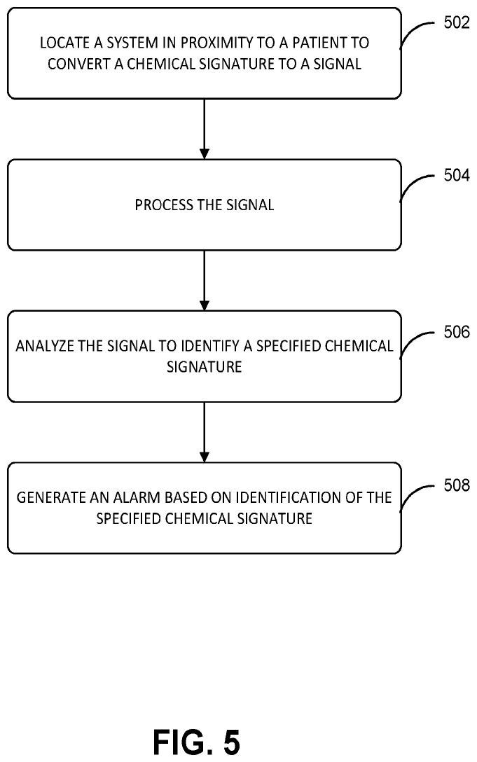

FIG. 5 shows an example method 500 for using the system 100, such as to identify a leak associated with an insulin pump, infusion set, or infusion site. In an example, the method 500 can be applied to identify an insulin leak associated with an insulin infusion site.

At block 502, the system 100 can be placed in proximity to a patient, such as the sensor 110 can be located on the patient near an infusion site. The sensor 110 can be selected to detect a specified chemical signature, such as a gaseous chemical or chemicals in an environment surrounding the patient. In an example, the specified chemical signature can include a chemical associated with the presence of insulin, such as an exogenous insulin used in the treatment of diabetes in the environment surrounding the patient. The sensor 110 can convert the chemical signature into a signal, such as an electrical signal representative of the specified chemical signature.

At block 504, the system 100 can process a signal, such as the electrical signal representative of the specified chemical signature. Processing the signal can include at least one of reading the signal, such as by receiving the signal from a sensor 110, or transmitting the signal, such as transmitting the signal to another component of the system 100. Processing the signal can include performing a mathematical operation on a signal, such as one or more signals. In an example, a mathematical operation can include at least one of addition, subtraction, multiplication, or division. Processing the signal can include setting a value, such as setting an alarm signal value for analysis.

At block 506, the system 100 can analyze the signal, such as to indicate the presence or concentration of a chemical or analyte defined by the specified chemical signature. Analyzing the signal can include analyzing the signal with a pattern recognition algorithm. In an example, the pattern recognition algorithm can include a filtering operation, such as by applying a Kalman filter (including a weighted Kalman filter) to the received signal. In an example, the pattern recognition algorithm can include an artificial intelligence (AI) operation, such as by applying an Al algorithm configured to indicate the presence of a chemical defined by the specified chemical signature from the received signal. In an example, the pattern recognition algorithm can include a machine learning algorithm.

Analyzing the signal can include setting an alarm signal value, such as to indicate the presence of a chemical identified through analysis of the received signal with a pattern recognition algorithm. In an example, the alarm signal value can assume a value of “0” should analysis of the received signal indicate the absence of the specified chemical signature or a value of “1” should analysis of the received signal indicate the presence of the specified chemical signature

At block 508, the system 100 can generate an alarm, such as based on the alarm signal value. Generating an alarm can include polling the alarm signal value, such as to ascertain a baseline operational state or detect a change in operational state. A change in operational state can indicate the presence of a leak, such as a leak of exogenous insulin from an insulin infusion site.

A first operational state of the system 100 can be represented by an alarm signal value of “0” such as indicating the absence of a specified chemical signature, including an exogenous insulin chemical signature. A second operational state can be represented by an alarm signal value of “1,” such as indicating the presence of a specified chemical signature. In an example, a transition of an alarm signal value of “0” to an alarm signal value of “1” can indicate the presence of the specified chemical signature, such as to indicate a leak from the patient infusion site.

Generating an alarm can include sending an alarm signal to the system 100, such as a component of the system 100. In an example, sending an alarm signal can include warning a user of a leak, such as by generating an audible alarm signal, by generating a visible alarm signal, or by generating a tactile alarm signal. In an example, sending an alarm signal can include sending an indication that a leak has occurred to an insulin pump. In an example, sending an alarm signal can include monitoring the alarm signal, such as streaming alarm signal data to a storage device for further subsequent data analysis. In an example, sending an alarm signal can include processing the alarm signal data, such as to diagnose or otherwise predict the cause of a leak based at least in part on the monitored alarm signal. In an example, sending an alarm signal can include generating an alert, such as a tactile alarm, to inform a patient of a potential infusion set issue. In an example, sending an alarm signal can include displaying additional information to the user, such as the time of the leak onset, the duration of the leak, the location of the leak, and the estimated quantity of medication not delivered.

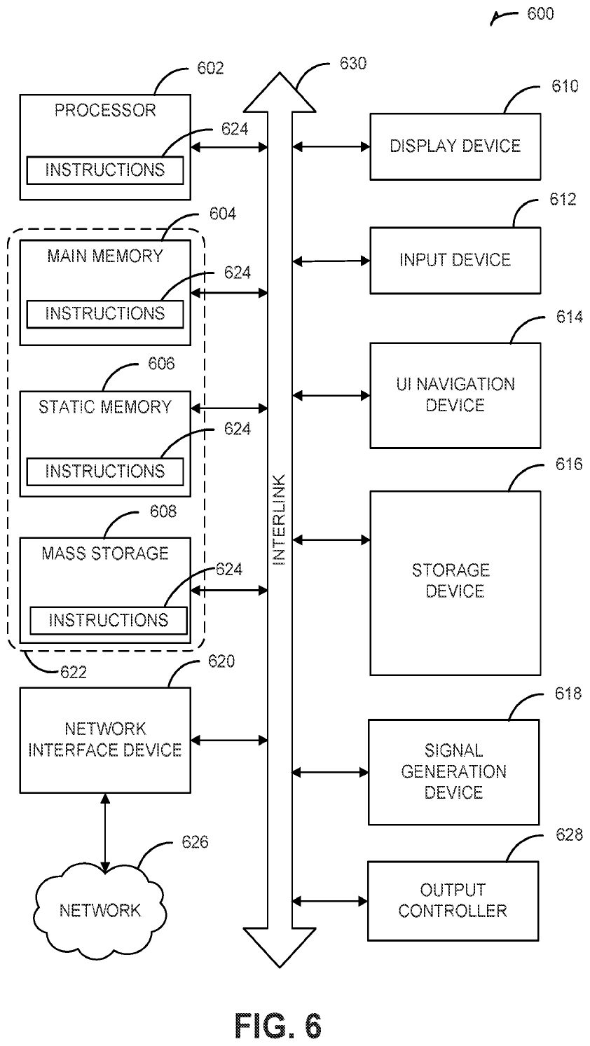

FIG. 6 illustrates a block diagram of an example machine 600 upon which any one or more of the techniques (e.g., methodologies) discussed herein may perform. The machine 600 may be a local or remote computer, or processing node in an “on-the-go” (OTG) device such as a smartphone, tablet, or wearable device. The machine 600 may operate as a standalone device or may be connected (e.g., networked) to other machines. In an embodiment, the machine may be directly coupled or be integrated with the system 100. It will be understood that when the processor 602 is coupled directly to the system 100, that some components of machine 600 may be omitted to provide a lightweight and flexible device (e.g., display device, user interface (UI) navigation device, etc.). In a networked deployment, the machine 900 may operate in the capacity of a server machine, a client machine, or both in server-client network environments. In an example, the machine 600 may act as a peer machine in peer-to-peer (P2P) (or other distributed) network environment. The machine 600 can include a personal computer (PC), a tablet PC, a microcontroller, a microprocessor, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

Examples, as described herein, may include, or may operate by, logic or a number of components, or mechanisms. Circuitry is a collection of circuits implemented in tangible entities that include hardware (e.g., simple circuits, gates, logic, etc.). Circuitry membership may be flexible over time and underlying hardware variability. Circuitries include members that may, alone or in combination, perform specified operations when operating. In an example, hardware of the circuitry may be immutably designed to carry out a specific operation (e.g., hardwired). In an example, the hardware of the circuitry may include variably connected physical components (e.g., execution units, transistors, simple circuits, etc.) including a computer readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuitry in hardware via the variable connections to carry out portions of the specific operation when in operation. Accordingly, the computer readable medium is communicatively coupled to the other components of the circuitry when the device is operating. In an example, any of the physical components may be used in more than one member of more than one circuitry. For example, under operation, execution units may be used in a first circuit of a first circuitry at one point in time and reused by a second circuit in the first circuitry, or by a third circuit in a second circuitry at a different time.

Machine (e.g., computer system) 600 may include a hardware processor 602 (e.g., a central processing unit (CPU), a graphics processing unit (GPU), a hardware processor core, or any combination thereof), a main memory 604 and a static memory 606, and a mass storage 608, some or all of which may communicate with each other via an interlink (e.g., bus) 630. The machine 600 may further include a display unit 610, an alphanumeric input device 612 (e.g., a keyboard), and a user interface (UI) navigation device 614 (e.g., a mouse). In an example, the display unit 610, input device 612 and UI navigation device 614 may be a touch screen display. The machine 600 may additionally include a storage device (e.g., drive unit) 616, a signal generation device 618 (e.g., a speaker), a network interface device 620, and one or more sensors 110. The machine 600 may include an output controller 628, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

The storage device 616 may include a machine readable medium 622 on which is stored one or more sets of data structures or instructions 624 (e.g., software) embodying or utilized by any one or more of the techniques or functions described herein. The instructions 624 may also reside, completely or at least partially, within the main memory 604, within static memory 606, or within the hardware processor 602 during execution thereof by the machine 600. In an example, one or any combination of the hardware processor 602, the main memory 604, the static memory 606, or the storage device 616 may constitute machine readable media.

While the machine readable medium 622 is illustrated as a single medium, the term “machine readable medium” may include a single medium or multiple media (e.g., a centralized or distributed database, or associated caches and servers) configured to store the one or more instructions 624.

The term “machine readable medium” may include any medium that is capable of storing, encoding, or carrying instructions for execution by the machine 600 and that cause the machine 600 to perform any one or more of the techniques of the present disclosure, or that is capable of storing, encoding or carrying data structures used by or associated with such instructions. Non-limiting machine-readable medium examples may include solid-state memories, and optical and magnetic media. In an example, a massed machine-readable medium comprises a machine-readable medium with a plurality of particles having invariant (e.g., rest) mass. Accordingly, massed machine-readable media are not transitory propagating signals. Specific examples of massed machine-readable media may include: non-volatile memory, such as semiconductor memory devices (e.g., Electrically Programmable Read-Only Memory (EPROM), Electrically Erasable Programmable Read-Only Memory (EEPROM)) and flash memory devices: magnetic disks, such as internal hard disks and removable disks: magneto-optical disks; and CD-ROM and DVD-ROM disks.

The instructions 624 may further be transmitted or received over a communications network 626 using a transmission medium via the network interface device 620 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as Wi-Fi®, IEEE 802.16 family of standards known as WiMax (R). IEEE 802.15.4 family of standards, peer-to-peer (P2P) networks, among others. In an example, the network interface device 620 may include one or more physical jacks (e.g., Ethernet, coaxial, or phone jacks) or one or more antennas to connect to the communications network 626. In an example, the network interface device 620 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding or carrying instructions for execution by the machine 600, and includes digital or analog communications signals or other intangible medium to facilitate communication of such software.

### Various Notes

The above description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B.” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Geometric terms, such as “parallel,” “perpendicular,” “round,” or “square,” are not intended to require absolute mathematical precision, unless the context indicates otherwise. Instead, such geometric terms allow for variations due to manufacturing or equivalent functions. For example, if an element is described as “round” or “generally round.” a component that is not precisely circular (e.g., one that is slightly oblong or is a many-sided polygon) is still encompassed by this description.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

1-9. (canceled)

10. A system for detecting a leak of a therapeutic drug at or near an infusion or injection site, the system comprising:
a sensor configured to detect at least one gaseous chemical emitted from the leaked therapeutic drug and to produce an electrical signal representative of the at least one gaseous chemical;
a microcontroller unit (MCU) coupled to the sensor, the MCU configured to receive and process the electrical signal; and
a communications unit coupled to the MCU, the communications unit configured to transmit the processed electrical signal to a user device to indicate detection of the leaked therapeutic drug.

11. The system of claim 10, wherein the leaked therapeutic drug comprises exogenous insulin.

12. The system of claim 11, wherein the at least one gaseous chemical is selected from the group consisting of: insulin lispro, insulin lispro-aabc, insulin aspart, fast-acting insulin aspart (faster aspart), insulin glulisine, insulin human (regular insulin), neutral protamine Hagedorn (NPH) or isophane insulin, protamine zinc insulin, insulin glargine, insulin glargine-yfgn, insulin detemir, insulin human inhalation powder, arginine hydrochloride, dibasic sodium phosphate, disodium hydrogen phosphate dihydrate, disodium phosphate dihydrate, endogenous zinc, fumaryl diketopiperazine, glycerin, glycerol, hydrochloric acid, L-arginine, liraglutide, lixisenatide, magnesium chloride hexahydrate, mannitol, metacresol (m-cresol), methionine, niacinamide (vitamin B3), phenol, polysorbate 20, polysorbate 80, protamine sulfate, sodium chloride, sodium citrate dihydrate, sodium hydroxide, treprostinil sodium, tromethamine, zinc, zinc acetate, zinc oxide, zinc chloride, water, and combinations thereof.

13. The system of claim 10, wherein the sensor comprises a plurality of sensing elements.

14. The system of claim 13, wherein each sensing element comprises at least one of a metal oxide semiconductor (MOS), a carbon material, a metal organic framework (MOF), a covalent organic framework (COF), a phyllosilicate, a conducting polymer (CP), a transitional metal dichalcogenide (TMDC), and Mxene.

15. The system of claim 13, wherein each sensing element is enclosed in, surrounded by, or in located in proximity to a perm-selective membrane configured to restrict adsorption of gaseous molecules onto the sensing element.

16. The system of claim 15, wherein the perm-selective membrane comprises at least one of a polymer barrier including a molecular imprinted polymer; an inorganic barrier, including metal oxides; a molecular organic framework (MOF) barrier; and/or a covalent organic framework (COF) barrier.

17. The system of claim 13, wherein each sensing element is defined by an electrical property selected from the group consisting of an electrical impedance, an electrical capacitance, an electrical resistance, and combinations thereof; and wherein adsorption of the gaseous molecules changes the electrical property.

18. The system of claim 10, wherein the sensor is located on an insulin infusion pump, cartridge, tubing, or cannula.

19. The system of claim 10, wherein the sensor is located on a patient's skin or is wearable by the patient.

20. The system of claim 10, wherein the MCU is configured to process the electrical signal by generating an algorithmic output.

21. The system of claim 10, wherein the MCU comprises a control module configured to control operation of, and communication between or within, the sensor, the MCU, and the communications unit.

22. The system of claim 10, wherein the MCU comprises an artificial intelligence (AI) module configured to process the electrical signal to identify, quantify, and/or characterize the leaked therapeutic drug.

23. The system of claim 22, wherein the MCU comprises an alarm module configured to receive input from the AI module and to output an alarm signal indicating a presence or absence of the leaked therapeutic drug.

24. The system of claim 23, wherein the alarm module is further configured to output a timing characteristic of the leaked therapeutic drug.

25. The system of claim 23, wherein the alarm signal is an audible, visual, or tactile signal.

26. The system of claim 10, wherein the user device comprises at least one of a medical device or a on-the-go device.

27. A method for detecting a leak of a therapeutic drug at or near an infusion or injection site, the method comprising:
sensing a signal indicative of at least one gaseous chemical emitted from the leaked therapeutic drug;
converting the sensed signal into an electrical signal;
processing the electrical signal to identify, quantify, and/or characterize the at least one gaseous chemical; and
generating an alarm based on the deviation of the at least one gaseous chemical from a threshold range.

28. The method of claim 27, wherein the leaked therapeutic drug comprises exogenous insulin; and wherein the at least one gaseous chemical is selected from the group consisting of: insulin lispro, insulin lispro-aabc, insulin aspart, fast-acting insulin aspart (faster aspart), insulin glulisine, insulin human (regular insulin), neutral protamine Hagedorn (NPH) or isophane insulin, protamine zinc insulin, insulin glargine, insulin glargine-yfgn, insulin detemir, insulin human inhalation powder, arginine hydrochloride, dibasic sodium phosphate, disodium hydrogen phosphate dihydrate, disodium phosphate dihydrate, endogenous zinc, fumaryl diketopiperazine, glycerin, glycerol, hydrochloric acid, L-arginine, liraglutide, lixisenatide, magnesium chloride hexahydrate, mannitol, metacresol (m-cresol), methionine, niacinamide (vitamin B3), phenol, polysorbate 20, polysorbate 80, protamine sulfate, sodium chloride, sodium citrate dihydrate, sodium hydroxide, treprostinil sodium, tromethamine, zinc, zinc acetate, zinc oxide, zinc chloride, water, and combinations thereof.

29. The method of claim 27, wherein processing the electrical signal comprises using a pattern recognition, artificial intelligence (AI), or machine learning algorithm.

30. A machine readable medium for detecting a leak of a therapeutic drug at or near an infusion or injection site, the machine readable medium storing, encoding, or carrying instructions for execution by a machine, the instructions for:
sensing a signal indicative of at least one gaseous chemical emitted from the leaked therapeutic drug;
converting the sensed signal into an electrical signal;
processing the electrical signal to identify, quantify, and/or characterize the at least one gaseous chemical; and
generating an alarm based on the deviation of the at least one gaseous chemical from a threshold range.

31. The machine readable medium of claim 30, wherein the leaked therapeutic drug comprises exogenous insulin; and wherein the at least one gaseous chemical is selected from the group consisting of: insulin lispro, insulin lispro-aabc, insulin aspart, fast-acting insulin aspart (faster aspart), insulin glulisine, insulin human (regular insulin), neutral protamine Hagedorn (NPH) or isophane insulin, protamine zinc insulin, insulin glargine, insulin glargine-yfgn, insulin detemir, insulin human inhalation powder, arginine hydrochloride, dibasic sodium phosphate, disodium hydrogen phosphate dihydrate, disodium phosphate dihydrate, endogenous zinc, fumaryl diketopiperazine, glycerin, glycerol, hydrochloric acid, L-arginine, liraglutide, lixisenatide, magnesium chloride hexahydrate, mannitol, metacresol (m-cresol), methionine, niacinamide (vitamin B3), phenol, polysorbate 20, polysorbate 80, protamine sulfate, sodium chloride, sodium citrate dihydrate, sodium hydroxide, treprostinil sodium, tromethamine, zinc, zinc acetate, zinc oxide, zinc chloride, water, and combinations thereof.

32. The machine readable medium of claim 30, wherein processing the electrical signal comprises using a pattern recognition, artificial intelligence (AI), or machine learning algorithm.
